(19) United States
(12) Patent Application Publication
(10) Pub. No.: US 2007/0244490 A1
(43) Pub. Date: Oct. 18, 2007

(54) Growth cuff removal devices and methods of use

(76) Inventors: Ryan T. Moehle, Salt Lake City, UT (US); Ryan C. Patterson, Flagstaff, AZ (US); Scott W. Sanders, Lehi, UT (US); Guy T. Rome, West Valley City, UT (US); J. William Poulson, Tooele, UT (US); Donald R. Weber, Ogden, UT (US); Betty Jean King, Magna, UT (US); Jeffery D. Bright, Stansbury Park, UT (US); William R. Barron, Riverton, UT (US); Kelly J. Christian, Draper, UT (US); Kenneth W. Sykes, Sandy, UT (US)

Correspondence Address: STOEL RIVES LLP - SLC, 201 SOUTH MAIN STREET, ONE UTAH CENTER, SALT LAKE CITY, UT, 84111, US

(21) Appl. No.: 11/709,401

(22) Filed: Feb. 22, 2007

Related U.S. Application Data
(60) Provisional application No. 60/775,651, filed on Feb. 22, 2006.

(51) Int. Cl. A61M 25/00

(52) U.S. Cl. 606/108

(57) ABSTRACT

Methods and apparatuses for facilitating removal of a catheter from a patient are provided. A catheter removal device is disclosed comprising a gripping mechanism with at least two members each member having a proximal end and distal end. The distal end of the gripping mechanism having opposing clamping members which, when closed, approximate at least a portion of the outer circumference of a catheter securement device. The proximal end of the gripping mechanism has two handle members each having a loop structure to accommodate the thumb or finger, for example, of a medical practitioner. A middle portion of the gripping mechanism has a shank member wherein the at least two members are coupled together at a pivot point. A distal end of the clamping members has a taper to allow for dilation of tissue up to the location of the catheter securement device.

### CROSS-REFERENCE TO RELATED APPLICATION

This application claims the benefit of U.S. Provisional Application No. 60/775,651, entitled “INGROWTH CUFF REMOVAL DEVICES AND METHODS,” filed Feb. 22, 2006, which is incorporated herein by reference in its entirety.

### BRIEF DESCRIPTION OF FIGURES

FIG. 1 shows a perspective view of a one embodiment of a catheter removal tool;

FIG. 2 shows another perspective view of the catheter removal tool of FIG. 1;

FIG. 3 shows a perspective view of one embodiment of a catheter removal tool;

FIG. 4 shows a perspective view of one embodiment of a clamping member;

FIG. 5 shows a perspective view of the clamping member of FIG. 4 clamped onto a catheter securement device;

FIG. 6 shows a perspective view of one embodiment of a clamping member;

FIG. 7 shows a perspective view of one embodiment of a catheter removal tool;

FIG. 8 shows a perspective view of one embodiment of a portion of a catheter removal tool;

FIG. 9 shows a perspective view of one embodiment of a catheter removal tool;

FIG. 10 shows a perspective view of a one embodiment of a portion of a catheter removal tool;

FIG. 11 shows a perspective view of one embodiment of a portion of a catheter removal tool;

FIG. 12 shows a perspective view of one embodiment of a portion of a catheter removal tool;

FIG. 13 shows a perspective view of one embodiment of a catheter removal tool;

FIG. 14 shows a perspective view of the catheter removal tool of FIG. 13 with a sharp member extended;

FIG. 15 shows a perspective view of the catheter removal tool of FIG. 14 partially circumscribing a portion of a catheter and abutted against a catheter securement device;

FIG. 16 shows a side view of one embodiment of a catheter removal tool partially circumscribing a catheter and disposed within a cavity of a body;

FIG. 17 shows a side view of one embodiment of a catheter and catheter securement device implanted within a cavity of a body;

FIG. 18 shows a side view of one embodiment of a catheter and catheter securement device implanted within a cavity of a body;

FIG. 19 shows a side view of the catheter of FIG. 18 with a catheter removal tool disposed thereon;

FIG. 20 shows the catheter removal tool of FIG. 19 as an inflatable member is inflated;

FIG. 21 shows the catheter removal tool of FIGS. 19 and 20 as the inflatable member continues to inflate;

FIG. 22 shows a side view of one embodiment of a catheter removal tool;

FIG. 23 shows a side view of one embodiment of a catheter and catheter securement device;

FIG. 24 shows another side view of the catheter and catheter securement device of FIG. 23;

FIG. 25 shows a side view of one embodiment of a catheter and catheter securement device;

FIG. 26 shows another side view of the catheter and catheter securement device of FIG. 25.

FIG. 27 shows a perspective of one embodiment of a catheter and a catheter securement device;

FIG. 28 shows another perspective view of the catheter and catheter securement device of FIG. 27;

FIG. 29 shows a perspective view of one embodiment of a catheter and a catheter securement device;

FIG. 30 shows a side view of one embodiment of a catheter and a catheter securement device;

FIG. 31 shows a side view of one embodiment of a catheter and a catheter securement device;

FIG. 32 shows a side view of one embodiment of a catheter and a catheter securement device;

FIG. 33 shows a perspective view of one embodiment of a catheter and a catheter securement device;

FIG. 34 shows a side view of the catheter and catheter securement device of FIG. 33;

FIG. 35 shows a perspective view of one embodiment of a catheter and a catheter securement device;

FIG. 36 shows a side view of one embodiment of a catheter and a catheter securement device;

FIG. 37 shows a side view of one embodiment of a catheter and a catheter securement device;

FIG. 38 shows a side view of one embodiment of a catheter and a catheter securement device;

FIG. 39 shows a side view of one embodiment of a catheter and a catheter securement device;

FIG. 40 shows a side view of one embodiment of a catheter and a catheter securement device;

FIG. 41 shows a side view of one embodiment of a catheter and a catheter securement device;

FIG. 42 shows another side view of the catheter and catheter securement device of FIG. 41;

FIG. 43 shows a side view of one embodiment of a catheter and a catheter securement device;

FIG. 44 shows a perspective view of one embodiment of a catheter and a catheter securement device; and

FIG. 45 shows another perspective view of the catheter and catheter securement device of FIG. 44.

### DESCRIPTION OF THE INVENTION

The present invention relates to methods and apparatuses for facilitating removal of a catheter from a patient. Catheters have been developed for many different applications. Tunneled central venous catheters provide an important means of long-term venous access for many therapies, including, without limitation, parenteral nutrition, chemotherapy, and antibiotic therapy, as well as hemodialysis and plasmapheresis. Many catheters have a cuff attached to the catheter that, during catheter placement, is positioned in the body of a patient. The cuff allows growth of fibrous tissue that anchors the catheter to prevent accidental removal. Because fibrous tissue has grown around the cuff, removal of the catheter may sometimes be difficult. When the catheter is no longer required, removal of the catheter from the patient should cause the least amount of trauma as possible to the patient.

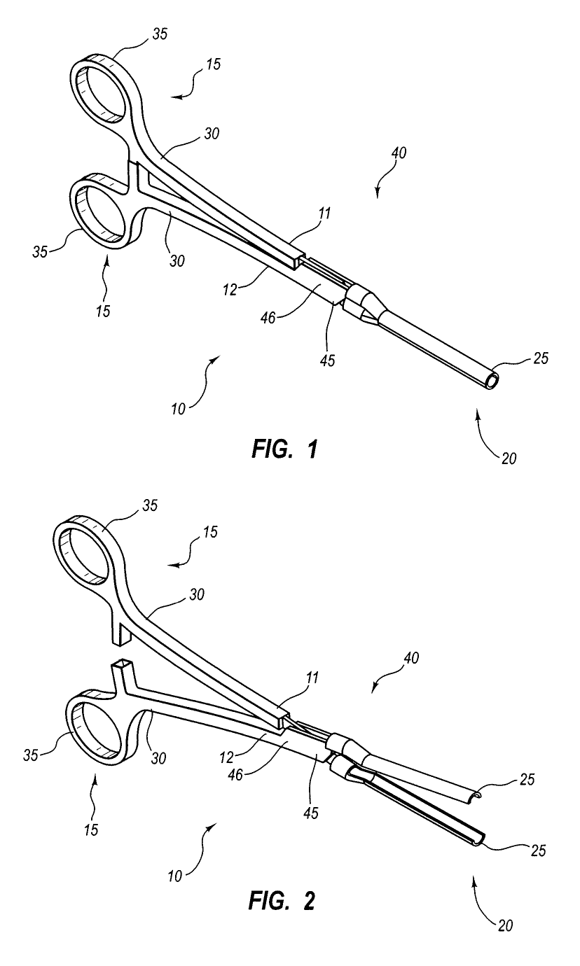

Referring generally to FIGS. 1-5, in one embodiment of the present invention, a device for removing a catheter 5 and catheter securement device 6 (e.g., a cuff) comprises a gripping mechanism 10 with at least two members (11,12). A distal end 20 of the gripping mechanism 10 has opposing clamping members 25 which, when closed, approximate at least a portion of the outer circumference of a catheter securement device 6. A proximal end 15 of the gripping mechanism 10 has two handle members 30 each having a loop structure 35 to accommodate the thumb or finger, for example, of a medical practitioner. A middle portion 40 of the gripping mechanism 10 has a shank member 45 wherein the at least two members (11,12) are coupled together at a pivot point 46. The gripping mechanism 10 is configured such that when the handle members 30 are pressed together, the clamping members 25 are also closed together. In another aspect, the gripping mechanism 10 is configured such that when the handle members 30 are pressed together, the clamping members 25 are opened. In one embodiment, an outer diameter of the clamping members 25 is configured to not substantially exceed the diameter of the catheter securement device 6. In another aspect, a distal end of the clamping members 25 has a taper to allow for dilation of tissue up to the location of the catheter securement device 6. In yet another embodiment, the clamping members 25 are disposed in a non-collinear but parallel plane to the handle members 30 so as to facilitate the closure of the gripping mechanism 10 while the clamping member 25 closes over the catheter securement device 6. In one aspect of the invention, a medical practitioner places the clamping members 25 over a portion of the catheter securement device 6 and forcibly removes the catheter securement device 6 and the catheter 5 from the body, separating the tissue growth from the catheter securement device 6.

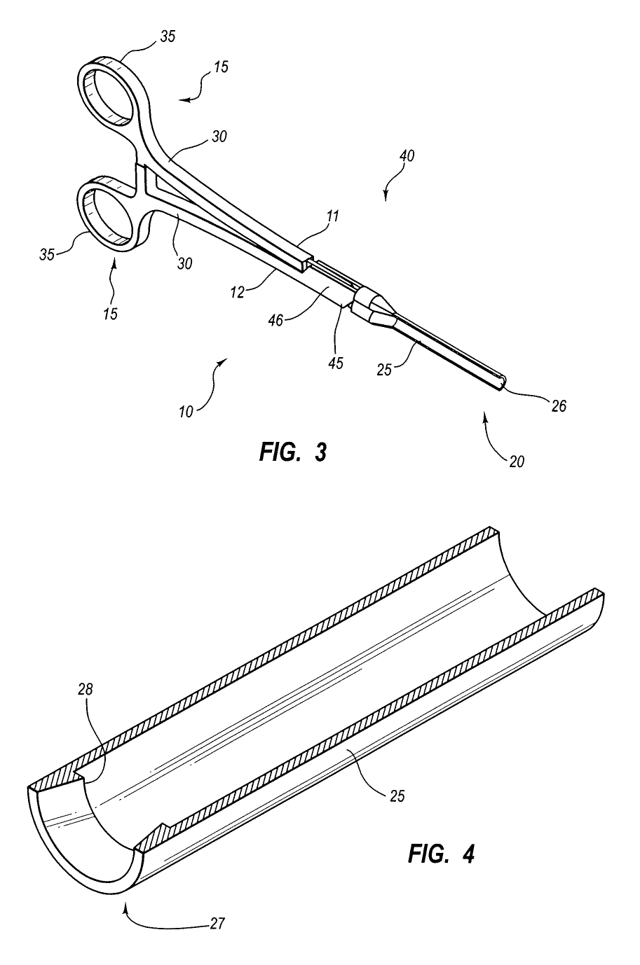

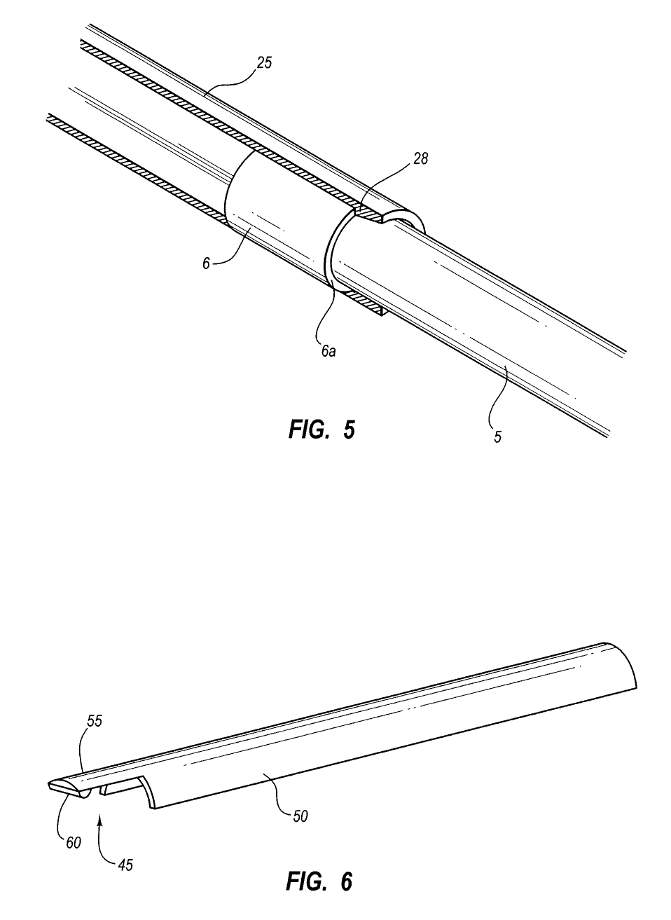

As illustrated in FIG. 3, in one embodiment, the clamping members 25, when closed, approximate a semi-circle 26. Referring now to FIGS. 4 and 5, in yet another embodiment, the distal end 27 of the clamping members 25 further comprises a radial protrusion 28 on an inner circumference of the clamping members 25. Advantageously, when the distal end 27 of the clamping members 25 is inserted over the catheter securement device 6 the radial protrusion 28 clamps over a distal end of the catheter securement device 6 thereby facilitating forcible removal of the catheter 5 and catheter securement device 6 from the patient. As illustrated in FIG. 6, in one aspect, a distal end 45 of at least one clamping member 50 may be provided with a longitudinally extending quarter-round section 55 with a radial protrusion 60 disposed thereon. Advantageously, the distal end of the quarter-round section may be rounded to minimize snagging of the catheter securement device during advancement of the tool over the catheter securement device. In another aspect, the distal end 45 of the clamping members 25 may be provided with depth markings to assist the medical practitioner in assessing the depth of the catheter securement device 6 within the patient.

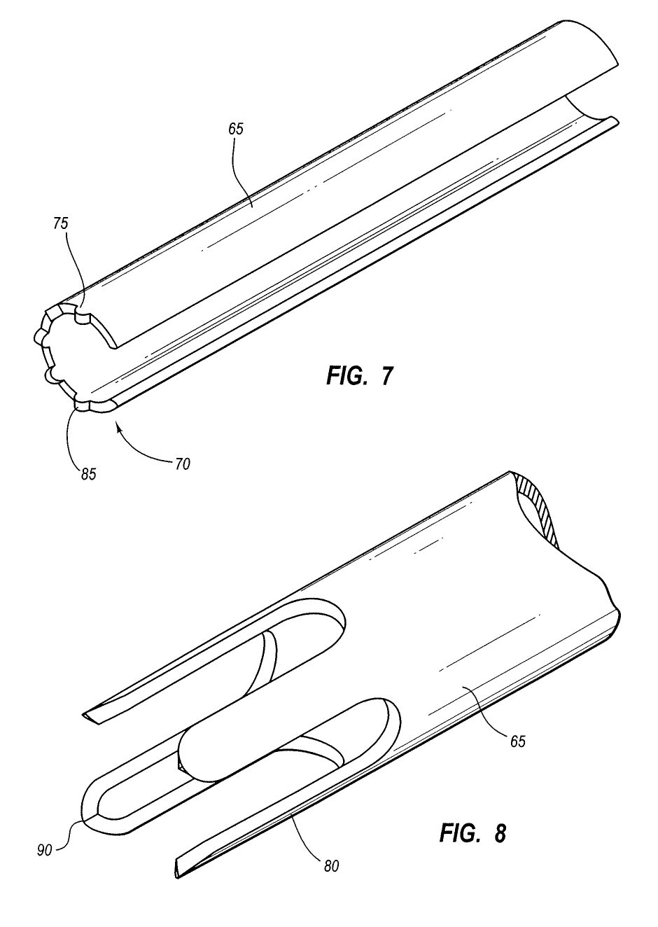

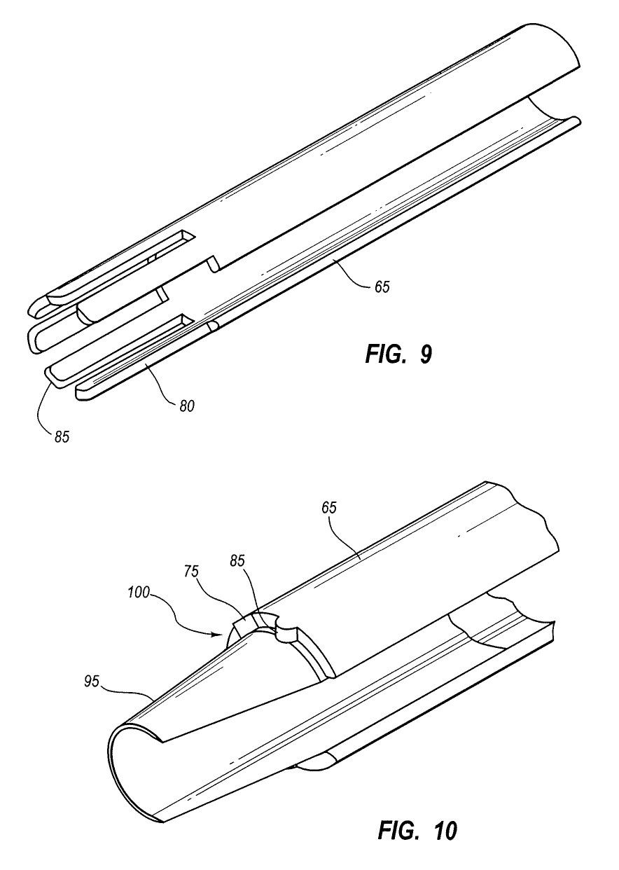

Referring now to FIGS. 7-10, in another embodiment of the present invention, a tool for facilitating removal of a catheter from a patient comprises a longitudinally extending arcuate member 65 shaped to approximate at least a portion of a catheter. In one aspect of the invention, the distal end 70 of the tool has a chamfered edge which separates tissue growth from the outside of a catheter securement device. In another aspect, a distal end 70 of the tool has at least one protrusion 75 disposed thereon for separating tissue growth from the catheter securement device. In one aspect of the invention, the medical practitioner places the tool over the catheter and advances the tool down the catheter until it abuts the body of the patient (e.g., where the catheter enters the body). The tool is sized such that the inner diameter of the tool is approximately equal to the outer diameter of the catheter securement device. The medical practitioner thereafter advances the tool over the catheter securement device. In one aspect of the invention, the medical practitioner twists and turns the tool as the tool is advanced over the catheter securement device in an effort to facilitate separation of the tissue growth from the catheter securement device. Once the tissue has been separated from the catheter securement device, the medical practitioner may remove the catheter from the patient. In one embodiment, the protrusion 75 comprises at least one semi-circle. In another embodiment, the protrusion further comprises at least one finger 80. In one aspect of the invention, as illustrated in FIGS. 8 and 9, the distal end of the tool comprises a plurality of finger-like protrusions 80 to facilitate removal of tissue growth from the catheter securement device. The protrusions may have blunt ends 80 (as shown in FIGS. 7 and 9) or sharpened ends 90 (as shown in FIG. 8) to optimize separation of tissue growth from the catheter securement device. The protrusions may also be tapered or beveled. Further, a tool may comprise various combinations of the above features (e.g., protrusions having blunt ends 80 and protrusions having sharp ends 90 on the same tool).

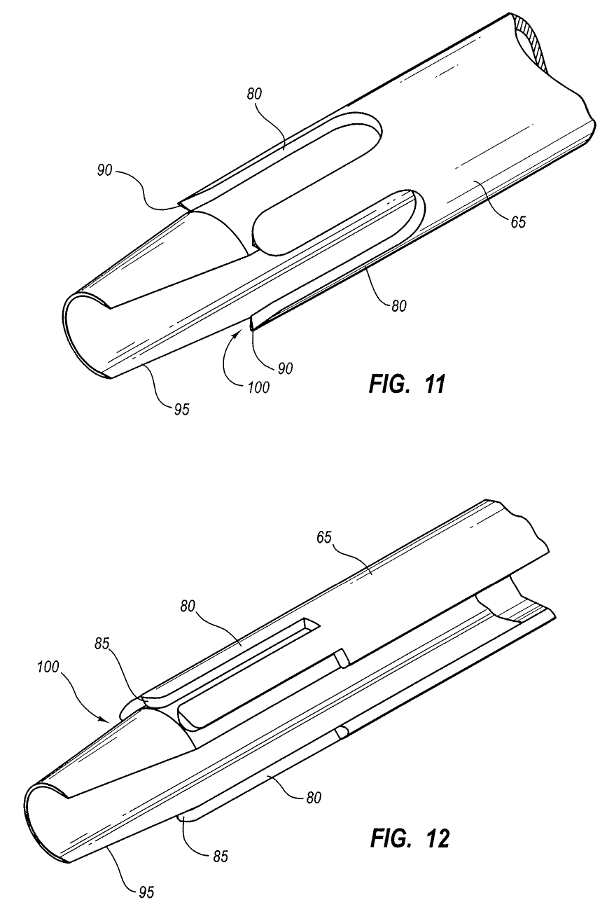

Referring now to FIGS. 10-12, in an additional embodiment, the tool further comprises an inwardly tapered member 95 extending past the distal end 100 of the protrusions (75, 80). Advantageously, the inwardly tapered member 95 dilates tissue around the catheter facilitating contact between the protrusions (75, 80) and the tissue growth around the catheter securement device. In one aspect of the invention, the inwardly tapered member 95 is removably coupled to the tool such that once tissue surrounding the catheter has been dilated and contact between the tool and the catheter securement device has been achieved, the inwardly tapered member 95 may be removed.

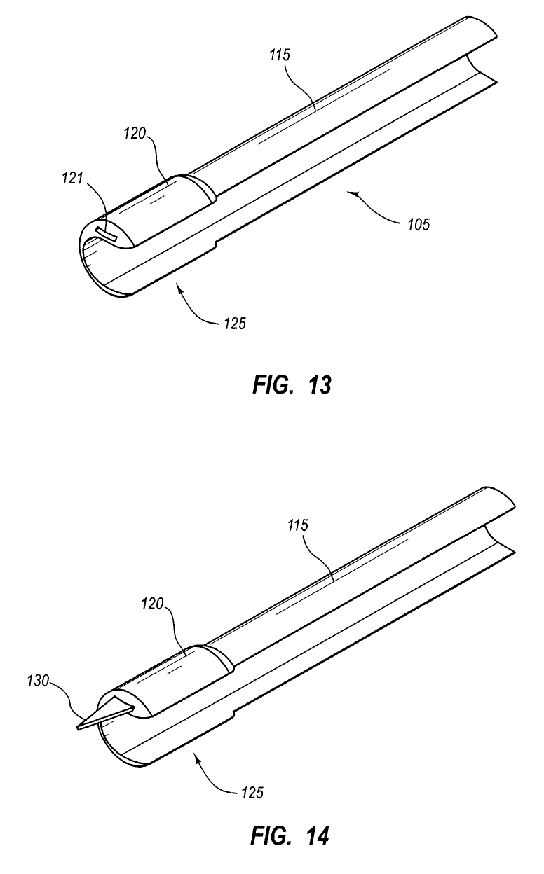

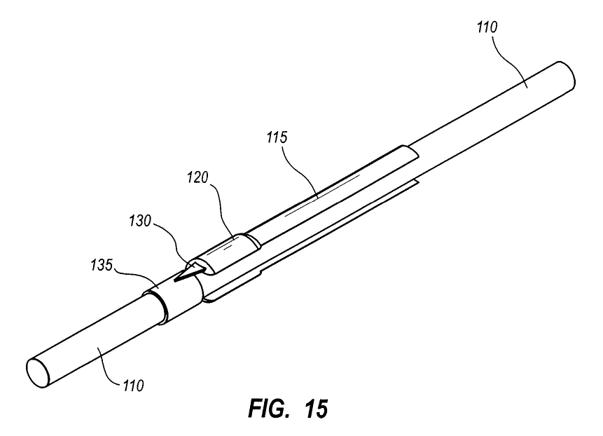

Referring now to FIGS. 13-15, in another embodiment, a tool 105 for facilitating removal of a catheter 110 from a patient comprises a longitudinally extending arcuate member 115 configured to at least partially circumscribe at least a portion of a catheter 110. A housing 120 is disposed on a distal end 125 of the tool 105 having a sharp member 130 (e.g., a razor) disposed therein. In one aspect of the invention, the sharp member 130 is retractably disposed within the housing 130. In another aspect, the sharp member 130 is fixed and the housing 120 is retractable to expose the sharp member 130. An aperture 121 is provided wherein the sharp member 130 may exit the housing 120. The tool 105 is configured such that when the tool 105 is advanced over the catheter 110, thereby abutting a catheter securement device 135, the sharp member 130 is extended to cut tissue growth away from the catheter securement device 135. The tool 105 is rotatable about a central axis of the catheter 110 to cut tissue away from the entire circumference of the catheter securement device 135. In another aspect of the invention, the housing 120 further comprises a threaded portion which mates to a corresponding threaded portion disposed on the tool 105. As the housing 120 threads around the corresponding threads on the tool 105, the housing 120 is advanced a predetermined distance which corresponds to the thread profile (e.g., the specific thread pitch, diameter, length, and depth) of the tool 105 and the housing 120.

Referring now to FIG. 16, in one embodiment, a tool 140 for removing a catheter 150 and a catheter securement device 160 is disclosed comprising a longitudinally extending arcuate member 170 separated into at least two portions connected to each other by a living hinge. In one aspect of the invention, a distal end 180 and a proximal end 190 of the tool 140 each have a tapered member (180a, 190a) disposed thereon. In one embodiment, an outer diameter of the tapered member 180a on the distal end 180 is smaller than an outer diameter of the tapered member 190a on the proximal end 190 of the tool 140. In one aspect, a medical practitioner clamps the tool 140 around a catheter 150 and advances the tool 140 down the catheter 150 until the distal end 180 abuts the site where the catheter enters/exits a patient. The medical practitioner advances the distal end 180 of the tool over the catheter 150 thereby physically separating the tissue from around the catheter 150. The medical practitioner thereafter withdraws the tool 140 and unclamps the tool 140. The medical practitioner then clamps the tool 140 back on the catheter 150 with the proximal end 190 facing the patient wherein the larger circumference tapered member 190a may be advanced over the catheter securement device 160 to further separate tissue growth from the catheter securement device 160.

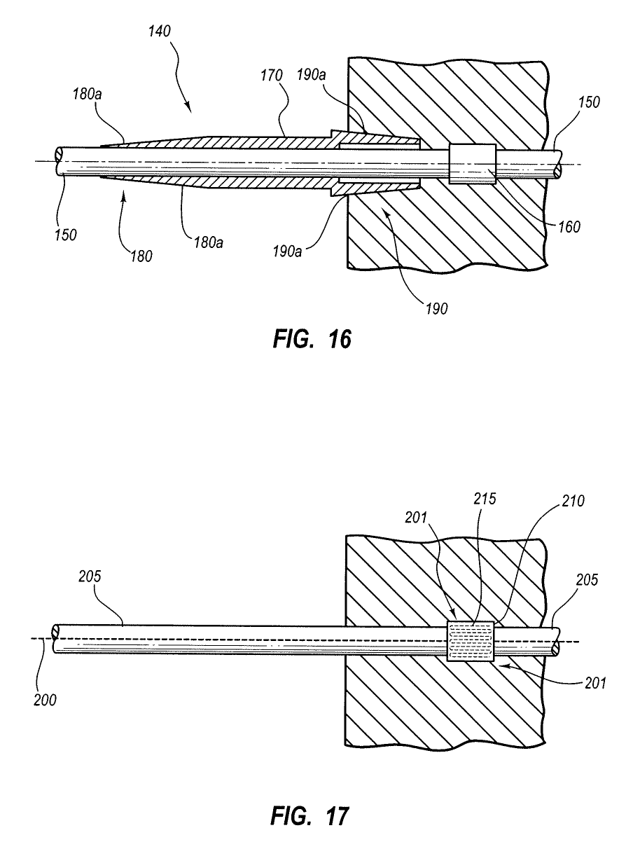

Referring now to FIG. 17, in another embodiment of the present invention, at least one conductive element 200 is disposed within a catheter 205 wherein the proximal end of the at least one conductive element 200 terminates at a distal opening of the catheter 205. Further, the distal end 215 of the at least one conductive element 200 is embedded within a catheter securement device 210. In one aspect of the invention, the distal end 215 of the conductive element 200 is embedded within the catheter securement device 210 such that the conductive element 200 separates the catheter securement device 210 into an inner portion and an outer portion. In one aspect, the portion of the conductive element 200 disposed within the catheter 205 is insulated and the portion of the conductive element 200 disposed within the catheter securement device 210 is not insulated. When a current (e.g., direct or alternating current) is applied to the conductive element 200, the portion that is not insulated heats the area surrounding the conductive element to at least the melting point of the surrounding catheter securement device 210 thereby separating the outer portion of the catheter securement device 210 from the inner portion of the catheter securement device 210. When an explantation force is applied by a medical practitioner, the catheter 205 can be removed as the conductive element 200 has separated the outer portion of the catheter securement device 210 from the inner portion of the catheter securement device 210. In another aspect of the invention, when current is applied to the conductive element 200, the tissue 201 surrounding the element 200 is heated to a suitable temperature (e.g., less than the melting point of the surrounding material). As the tissue 201 and the catheter securement device 210 are heated, the tissue 201 is loosened from around the catheter securement device 210 thereby minimizing the amount of stresses that must be overcome to break the bond between the tissue 201 and the catheter securement device 210.

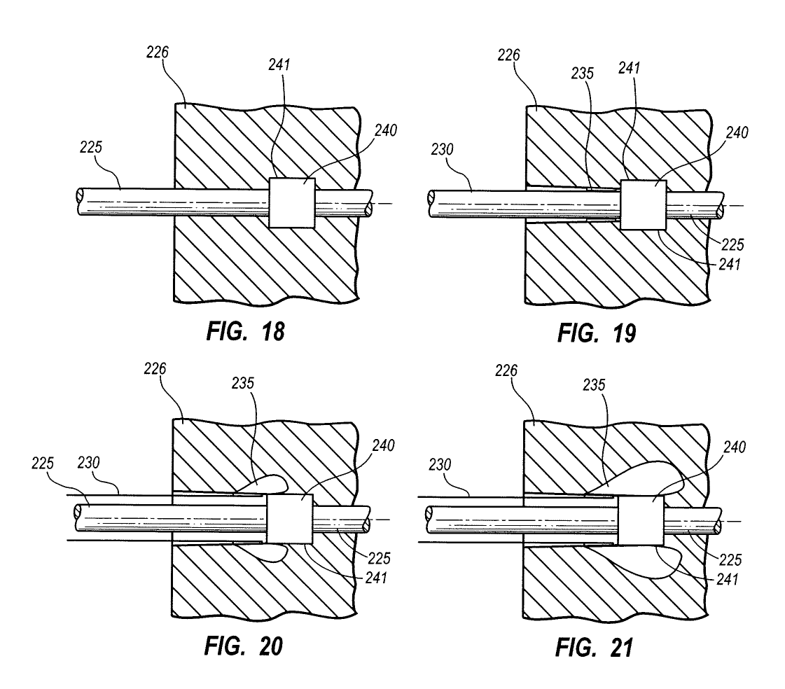

In another embodiment of the invention, as illustrated in FIGS. 18-21, a tool for facilitating removal of a catheter 225 from a patient 226 comprises a cylindrical body 230 configured to at least partially circumscribe a portion of a catheter 225. The cylindrical body 230 has an inflatable member 235 (e.g., balloon) disposed on a distal end thereof and a fluid pathway extending along the longitudinal body of the catheter 225. The fluid pathway is in fluid communication with the inflatable member 235. In one aspect, the tool is advanced over the catheter 225 until it abuts, or is adjacent to, a catheter securement device 240. The inflatable member 235 is configured, such that when it inflates, it inflates away from the distal end of the tool applying stress across the tissue growth/catheter securement device interface 241. As fluid is injected through the fluid pathway and into the inflatable member, the stress on interface 241 from the inflatable member 235 breaks the bond between the tissue growth and the catheter securement device 240, allowing the catheter 225 to be removed. In another embodiment, a catheter is provided with a fluid pathway communicating with a balloon disposed within the catheter securement device. The balloon is configured such that when it inflates, it radially expands the catheter securement device. As the catheter securement device expands radially (e.g., an increase in outer circumference of the catheter securement device), a stress is induced at the tissue growth/catheter securement device interface which breaks the bond between the tissue growth and the catheter securement device thereby facilitating removal of the catheter from the patient.

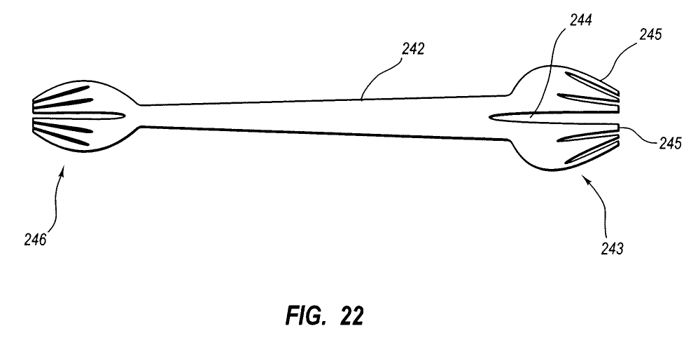

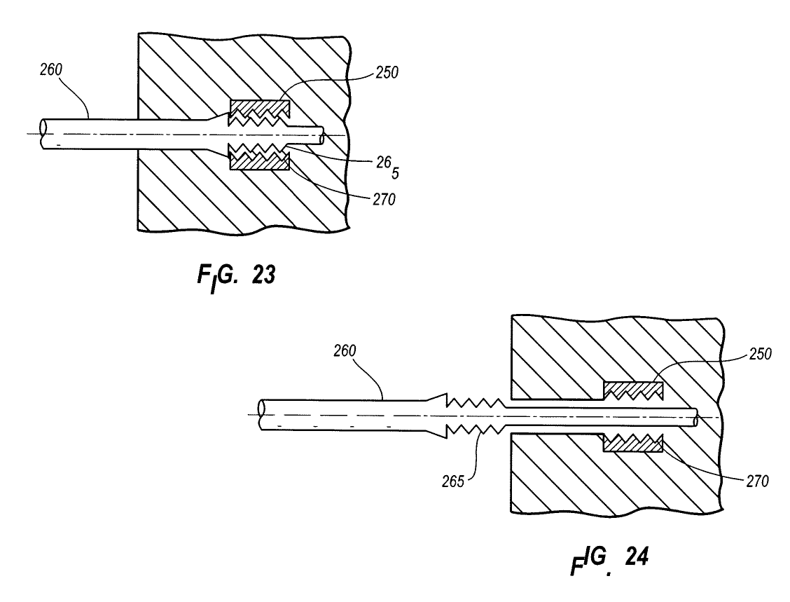

Referring to FIG. 22, in another embodiment, a device for removing a catheter securement device from a patient comprises a longitudinally extending arcuate member 242 configured to at least partially circumscribe a portion of a catheter. A distal end 243 of the device has a cavity 244 configured to receive a catheter securement device therein. Further, a plurality of finger members 245 is disposed on the distal end of the device, wherein the finger members 245 are biased to at least partially enclose the cavity 244. In one aspect, a medical practitioner advances the device over a catheter and subsequently over a catheter securement device using the finger members 245 to separate tissue growth from the catheter securement device while the cavity 244 receives the catheter securement device therein. As the medical practitioner advances the device over a distal end of the catheter securement device, the biased finger members 245 enclose the catheter securement device within the cavity 244. The medical practitioner thereafter removes the catheter and catheter securement device from the patient. In one aspect of the invention, a proximal end 246 of the device has a similar cavity and biased finger configuration as the distal end 243 but with larger or smaller dimensions.

In another embodiment, vibration is utilized as a means for removing a catheter from a patient. As noted above, catheter securement devices stimulate growth of fibrous tissue that anchors the catheter within the body of the patient, preventing accidental removal. Because fibrous tissue has grown around the catheter securement device, removal of the catheter may sometimes be difficult. In one embodiment of the present invention a vibrating device is attached to a portion of a catheter. The vibration device transfers vibrations throughout the catheter which loosens the tissue growth around the catheter securement device thereby facilitating removal of the catheter. In another aspect, a vibration device is applied directly to the body of the patient near the exit site of the catheter and the catheter securement device in order to loosen the tissue growth around the catheter securement device. It should be understood that the instant disclosure contemplates use of any device capable of generating vibrations (e.g., ultrasound, mechanical oscillation, etc.).

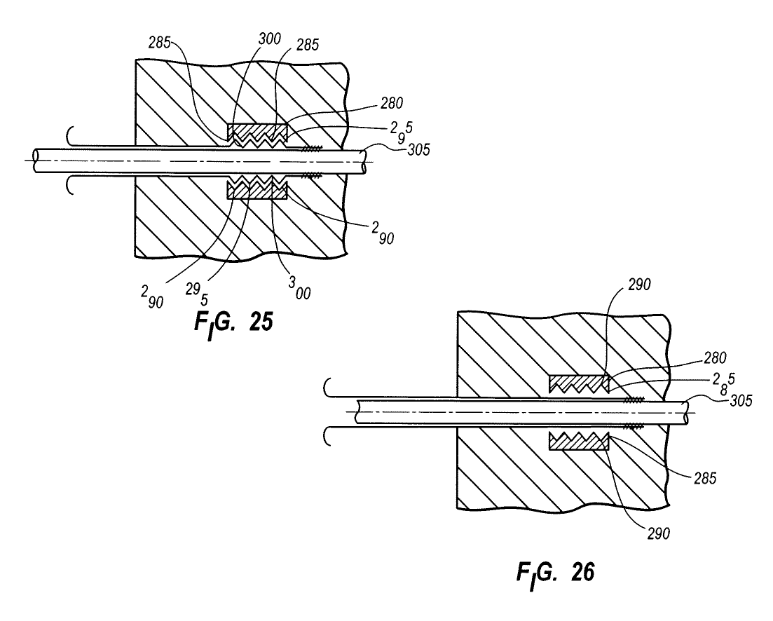

Referring now to FIGS. 23 and 24, in another embodiment of the present invention, a catheter securement device 250 is disclosed comprising an annular member 255 configured to at least partially circumscribe a catheter 260. In one aspect, the annular member 255 comprises a bio-absorbable material detachably connected to a catheter 260. The connection between the annular member 255 and the catheter 260 can be made by any suitable means wherein the catheter 260 and the catheter securement device 250 are disconnected at some future time. One example of a connection includes, without limitation, a threaded portion 265 on an outer wall of the catheter 260 configured to receive a corresponding threaded portion 270 disposed on the catheter securement device 250. An additional example of a connection includes at least one protrusion on the catheter and a mating groove corresponding to the protrusion on the catheter securement device. It should be understood that the inverse of the above aspect is also contemplated herein (e.g., catheter securement device having protrusion and catheter having corresponding mating groove). In this aspect of the invention, when the catheter 260 is to be removed from a patient, a medical practitioner disconnects the catheter 260 from the catheter securement device 250, and removes the catheter 260 from the patient. The catheter securement device 250 is left inside the body of the patient due to its bio-absorbable properties.

Referring to FIGS. 25 and 26, in another aspect, a bio-absorbable catheter securement device 280 comprises a plurality of ridges 285 and valleys 290 on an inner surface of the catheter securement device 280. The ridges 285 and valleys 290 of the catheter securement device 280 correspond to ridges 295 and valleys 300 disposed on an outer surface of a catheter 305 which lock the catheter 305 within the catheter securement device 280. Advantageously, the ridges 295 and valleys 300 disposed on the outer surface of the catheter 305 are configured to collapse when subjected to axial forces. Accordingly, once collapsed, the locking feature is eliminated and the catheter 305 is free to be removed from the body.

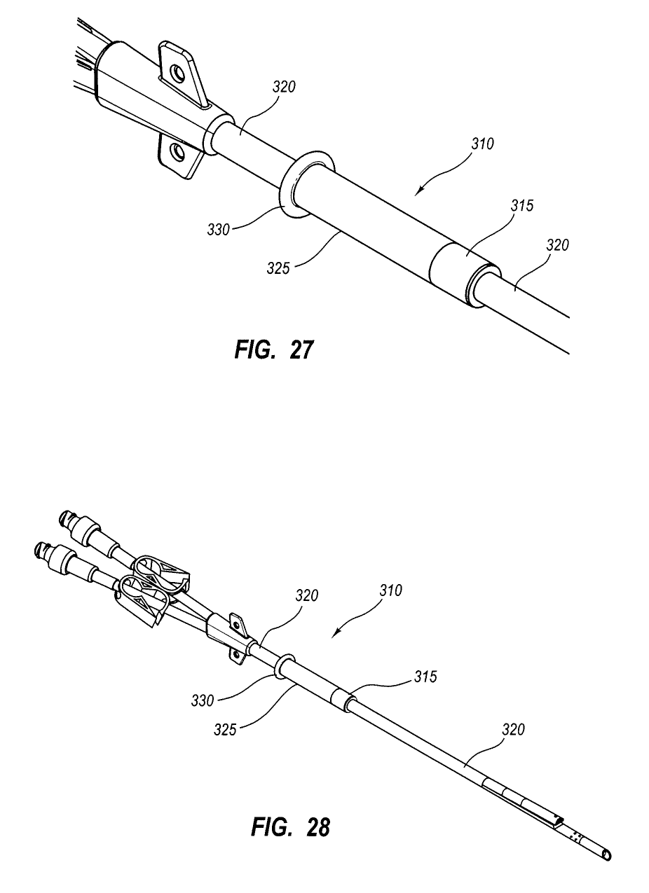

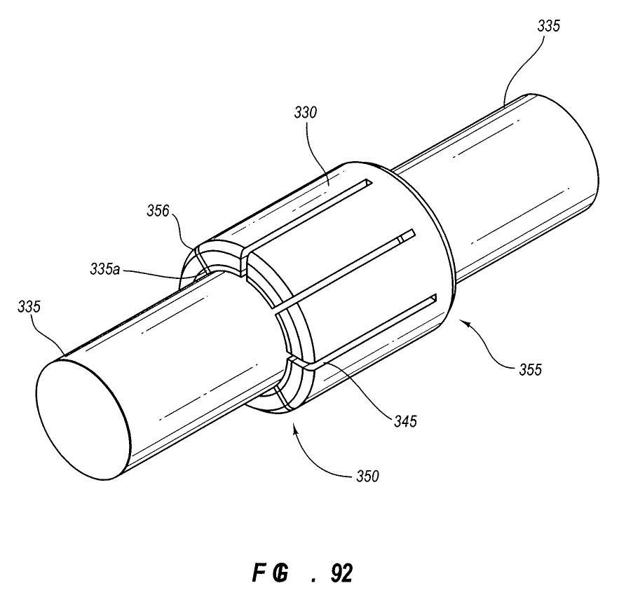

As illustrated in FIGS. 27 and 28, in another embodiment, a catheter securement device 310 is provided comprising an annular member 315 configured to at least partially circumscribe a catheter 320. The catheter securement device 310 further comprises a longitudinally extending member 325 which is also configured to at least partially circumscribe a catheter 320. When implanted within a portion of a patient, the catheter securement device 310, as noted above, acts as an anchor and biological seal for the catheter 320. In one aspect of the invention, the longitudinally extending member 325 extends outside of the patient having a gripping portion 330 disposed thereon. During explantation of the catheter 320, a medical practitioner grips the longitudinally extending member 325 and applies an explantation force in an attempt to remove the catheter 320 from a patient. Advantageously, the explantation force is exerted, via the longitudinally extending member 325, directly to the catheter securement device 310. In this manner, explantation forces are not distributed through the body of the catheter 320 causing the body of the catheter 320 to elongate. The longitudinally extending member 325 may be formed with the catheter securement device 310 or connected to the catheter securement device 310 by any suitable means (e.g., welded, fused, threaded, or pinned). The longitudinally extending member 325 may be manufactured from any suitable material including, without limitation, polymers, thermoplastics and elastomers. In another aspect of the invention, the shaft of the catheter 320 is reinforced (e.g., using fibers, wires, filaments, or other suitable means) in an area near the proximal end of the catheter securement device 310. Similar to above, when an explantation force is exerted on the reinforced area, the forces are not distributed throughout the body of the catheter 320 causing it to elongate, rather, the reinforcement directs the explantation load more directly to the catheter securement device 310.

Referring now to FIG. 29, in yet another embodiment, a catheter securement device comprises an annular member 330 at least partially circumscribing a catheter 335. In one aspect of the invention, the catheter securement device is bonded to the catheter 335 only at a distal end 340 of the securement device. The annular member 330 has at least one slit 345 extending parallel to a longitudinal axis of the catheter 335 from a proximal end 350 of the securement device to near the distal end 355 of the securement device. In another aspect of the invention, the annular member 330 has a plurality of slits 345 disposed about its outer circumference. In a direction perpendicular to the surface of the catheter, the slits 345 extend from the outer surface 356 of the securement device to the catheter 335a. As there is no bond between the catheter securement device and the catheter 335 where the slits are disposed, during explantation of the catheter 335 from a patient, the slits 350 allow the annular member 330 to peel away from the catheter 335. Advantageously, the movement of the annular member 330 away from the catheter 335 changes the stresses acting on the tissue growth/catheter securement device interface. When explanting a catheter with a securement device having no slits, for example, the primary stresses retaining the securement device are shear stresses related to the connection between the tissue growth and the securement device. When explanting a catheter having a securement device, as described above, the movement of the annular member away from the catheter body shifts the stresses acting on the annular member to peel stresses. In some cases, bonds are easier to break in a peel stress mode rather than a shear stress mode. In another aspect of the invention, the slits 345 extend nearly the entire length of the securement device. During explantation, rather than simply peel away from the catheter 335, the securement device is turned inside out. Similar to the above-noted aspect, peel forces would be the forces predominantly acting on the securement device.

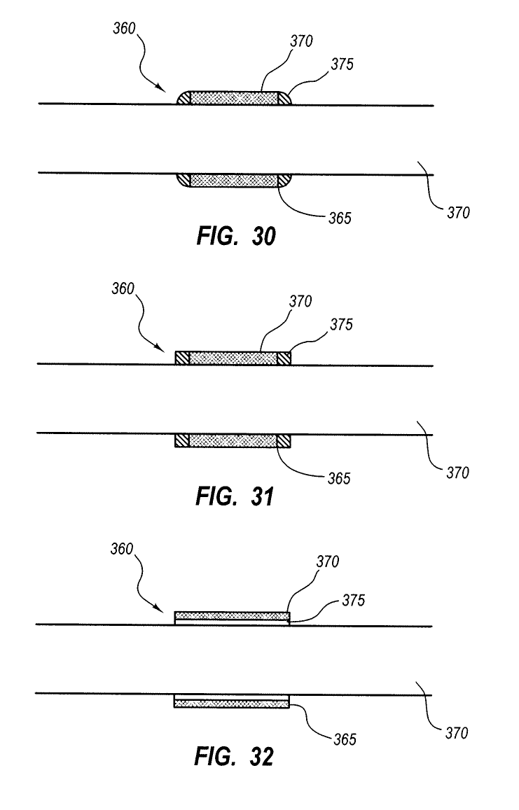

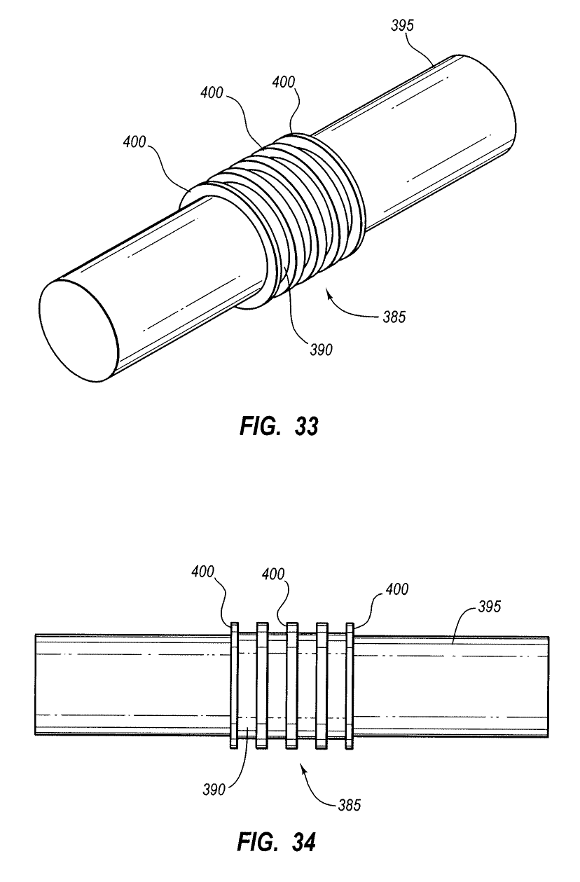

Referring generally to FIGS. 30-32, in yet another aspect of the present invention, a catheter securement device 360 comprises an annular member 365 configured to at least partially circumscribe a catheter 370. Tissue grows into a portion of the securement device 360 thereby minimizing accidental removal of the catheter 370 from the patient. As previously noted, the tissue growth around the securement device 360 provides a biological seal to minimize the potential for infections at the site where the catheter enters/exits the body of the patient. In one aspect, the annular member 365 comprises a first material 375 and at least a second material 380. The first material 375 comprises a non-absorbable, non-porous polymeric material (e.g., polyurethane). The second material 380 comprises an absorbable, porous material. The second material 380 comprises, for example, woven material, non-woven material, plant-based material, animal-based material, and synthetic material. After implantation within a patient, tissue grows into the absorbable material. Over time, the absorbable material degrades and the tissue fills in the vacancies left by the degrading absorbable material. In a catheter securement device wherein the securing tissue growth occurs within the pore spaces of a material with small pore spaces, for example, the mechanical connection between the tissue and the securement device is more difficult to separate due, at least in part, to the increased number of mechanical connections. The encapsulation provided for in the present invention would secure the catheter in place, yet advantageously, mechanically connect the tissue to the securement device in larger pore spaces facilitating easier removal of the securement device resulting in less trauma to the patient.

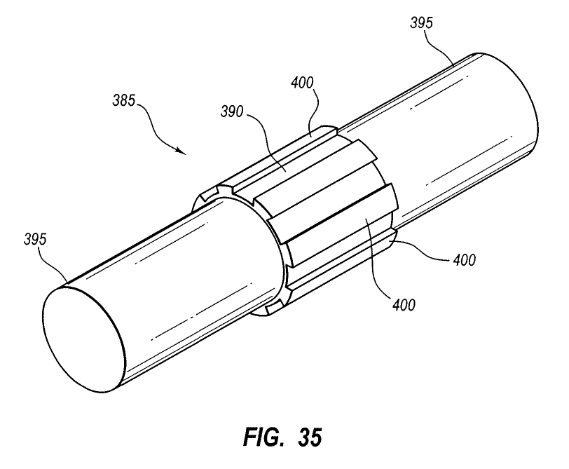

Referring now to FIGS. 33-35, in another embodiment of the invention, a catheter securement device 385 comprises an annular member 390 at least partially disposed about the outer circumference of a catheter 395. The annular member 390 further comprises a non-porous polymer having a plurality of ridges 400 formed thereon. In one aspect, the ridges 400 are parallel to the longitudinal axis of the catheter 395. In yet another aspect, the ridges 400 are perpendicular to the longitudinal axis of the catheter 395. The present disclosure contemplates, however, that the ridges 400 can be oriented in any direction relative to the longitudinal axis of the catheter 395.

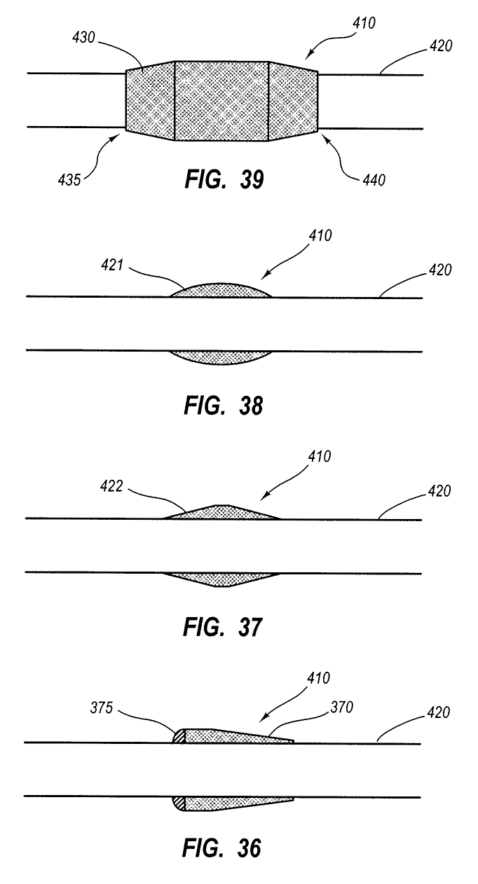

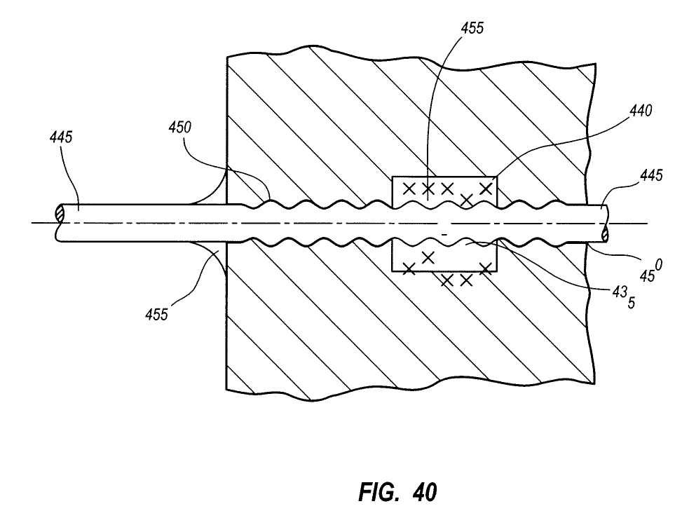

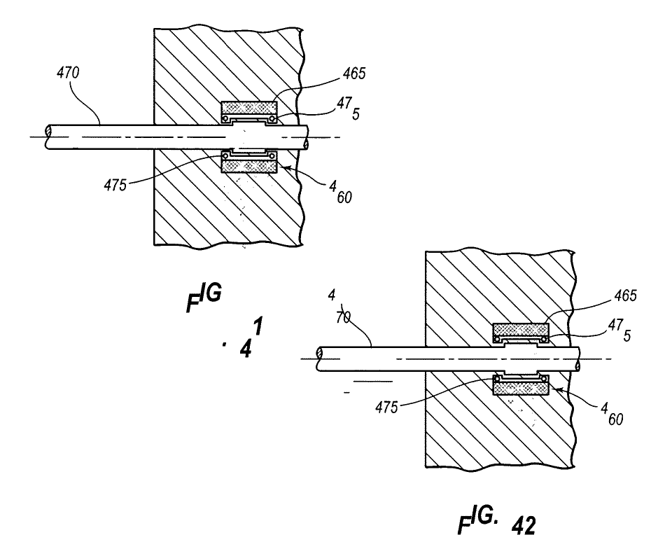

Referring generally to FIGS. 36-39, in another embodiment, a catheter securement device 410 comprises a substantially annular member 415 at least partially disposed about the outer circumference of a catheter 420. By shaping the surface in which tissue can grow in the catheter securement device 410, one can control the direction of tissue growth. In one aspect, referring now to FIG. 38, the catheter securement device 410 has a substantially arcuate profile 421 thereby maximizing shear stresses and minimizing stresses normal to the surface of the catheter securement device 410. In another aspect, as illustrated in FIG. 37, the catheter securement device 410 has an angled profile 422. In another embodiment of the invention, as shown in FIG. 39, a catheter securement device 410 has a mesh structure 430 disposed about the catheter securement device 410, the mesh structure 430 has a proximal end 435 and a distal end 440. In one aspect, the mesh structure 430 comprises a single strand of material. The single strand is configured such that when a user pulls on the strand of material on the proximal end 435 of the mesh structure 430, the entire mesh structure 430 unravels. Accordingly, any tissue growth incorporated within the mesh structure 430 is extricated with the mesh structure 430 or, in another aspect, tissue growth is left within the body. The unraveling of the mesh structure 430 breaks the bonds between the catheter securement device 410 and the tissue growth, allowing the catheter 420 to be removed from the patient.

Referring now to FIG. 40, in another embodiment, a catheter securement assembly is provided comprising a bio-absorbable annular member 440 configured to at least partially circumscribe a catheter 445. A plurality of ridges 450 are disposed on an outer surface the catheter 445. Bio-absorbable annular member 440 comprises a plurality of ridges 455 to mate with the ridges 450 disposed on the catheter 445. In one aspect, the bio-absorbable material is configured for incorporation into the body approximately 90 days after implantation. During the 90-day period, tissue grows around the ridges of the catheter to secure it in place. Once the bio-absorbable material is incorporated into the body, the tissue growth around the plurality of ridges 450 disposed on the catheter 445 continues to secure the catheter 445 into place. In another aspect of the invention, a temporary bonding agent (e.g., cyanoacrylate, Dermabond® medical sealant, Traumaseal® medical sealant) is utilized to fix the catheter in place by treating the catheter exit/entrance site with the agent as shown at 455. During the period in which the bonding agent disintegrates, tissue grows around the ridges 450 of the catheter 445 to secure it in place. Once the bonding agent is completely disintegrated, the tissue growth around the plurality of ridges 450 continues to secure the catheter 445 into place.

As illustrated in FIGS. 41 and 42, in another embodiment, a catheter securement device 460 is provided comprising an annular member 465 configured to at least partially circumscribe a catheter 470. The catheter securement device 460 further comprises a shape memory material (e.g., a shape memory alloy such as Nitinol) disposed within the catheter securement device 460. In one aspect of the invention, Nitinol bands 475 are disposed circumferentially within the annular member 465. Nitinol assumes different geometries when subjected to different temperature paradigms. In one aspect of the invention, the Nitinol band 475 assumes a constricted geometry while subjected to a temperature paradigm consistent with a typical internal temperatures of a patient. The constricted geometry fixes the catheter securement device 460 about the catheter 470. When the site surrounding the catheter securement device 460 is subjected to a different treatment paradigm (e.g., temperatures colder or hotter than typical internal temperatures of patient), the Nitinol band 475 contracts, thereby increasing the internal diameter of the catheter securement device 460. The contracted geometry releases the catheter 470 from the catheter securement device 460 whereafter the catheter 470 can be removed from the patient.

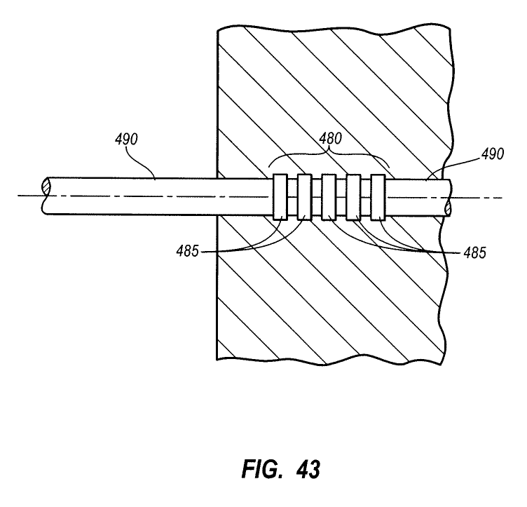

Referring now to FIG. 43, in another embodiment, a catheter securement device 480 is provided comprising a plurality of annular members 485 configured to at least partially circumscribe a catheter 490. The annular members 485 are longitudinally separated a predetermined distance 495 from one another along the body of the catheter 490. As a medical practitioner attempts to remove the implanted catheter 490, the force required to remove the catheter 490 from the patient is limited to the force to break the mechanical connection between one of the annular members 485 (e.g., the annular member nearest the exit site). Accordingly, the catheter 490 is removed as successive bonds are broken between the tissue growth and corresponding annular members 485.

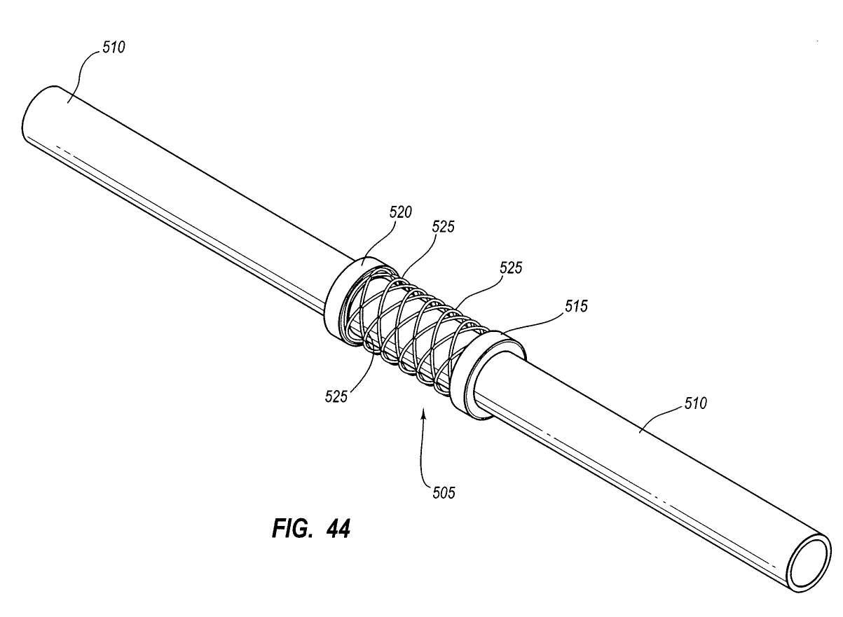

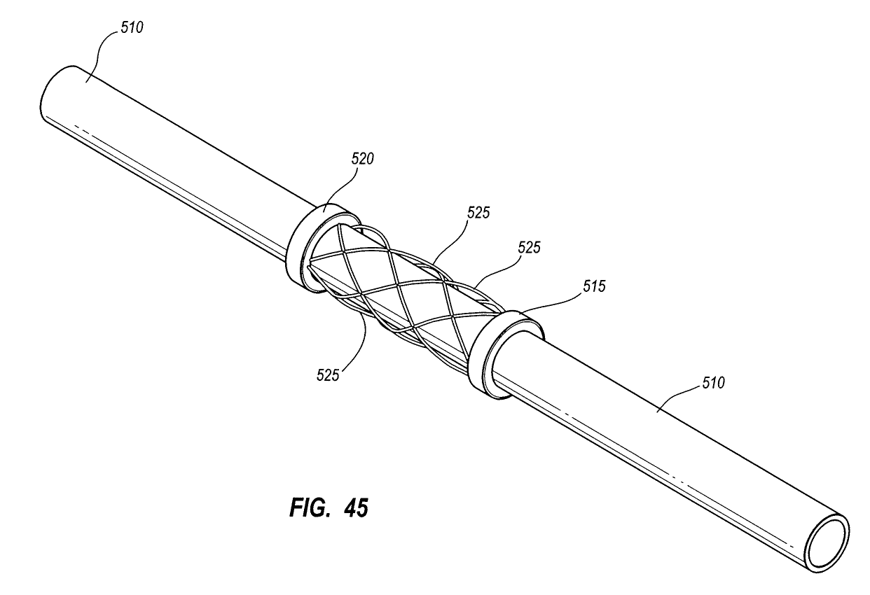

Referring to FIGS. 44 and 45, in another embodiment, a catheter securement device is provided comprising an annular member 505 configured to at least partially circumscribe a catheter 510. The annular member 505 comprises a distal band 515 and a proximal band 520 disposed on an outer circumference of the catheter 510. The distal band 515 and proximal band 520 have intertwining helical members 525 disposed therebetween. When the securement device 500 is implanted within a patient, tissue grows in the spaces between the helical members 525 thereby securing the catheter 510 within the patient. When it becomes desirable to remove the catheter 510, axial forces applied to the catheter 510 cause the helical members 525 to collapse. The collapsing action of the helical members 525 cuts the tissue growth from around the helical members 525 thereby allowing the catheter 510 to be removed from the patient. In one aspect of the invention, the helical members 525 comprise a rigid or semi-rigid material configured to dissect tissue when compressed against the tissue.

In another embodiment of the invention, a fluid is injected directly into a catheter securement device by a medical practitioner prior to explanation. The fluid is intended to weaken the bond between tissue growth and the catheter securement device. One example of such a fluid, without limitation, is lidocaine. When lidocaine is injected directly into the catheter securement device, the bond between tissue growth and the catheter securement device is weakened, thus facilitating removal of the catheter from a patient.

Although the above-described embodiments show a particular configuration of catheter securement devices, and methods and apparatuses for removing those devices, such embodiments are exemplary. Accordingly, many different embodiments are contemplated and encompassed by this disclosure. It should also be understood that the devices and methods for removing the catheter and catheter securement device can be used with any method or device wherein catheters are removed from a patient.

While certain embodiments and details have been included herein for purposes of illustrating aspects of the present invention, it will be apparent to those skilled in the art that various changes in the systems, apparatuses, and methods disclosed herein may be made without departing from the scope of the instant disclosure, which is defined, in part, in the appended claims. The words “including” and “having,” as used herein, including the claims, shall have the same meaning as the word “comprising.”

What is claimed is:

1. A device for removing a catheter from a patient comprising:
a gripping mechanism having a proximal end and a distal end;
an opposing pair of clamping members disposed on the distal end of the gripping mechanism, wherein the clamping members further comprise longitudinally extending arcuate members shaped to approximate at least a portion of an outer circumference of a catheter.

2. The device of claim 1, wherein at least one protrusion is disposed on an inner circumference of at least one clamping member.

3. The device of claim 2, wherein the at least one protrusion comprises a portion of an annular band disposed on the inner circumference of the at least one clamping member.

4. The device of claim 1, wherein the distal end of the device is disposed substantially parallel to the proximal end of the device, the distal end being further configured substantially non-collinear with the proximal end of the device.

5. A tool for removing a catheter securement device from a patient comprising:
a longitudinally extending arcuate member configured to at least partially circumscribe a portion of a catheter, the longitudinally extending arcuate member having a proximal end and a distal end; and
a cutting mechanism disposed on the distal end of the longitudinally extending arcuate member.

6. The tool of claim 5, wherein the cutting mechanism is retractably disposed on the distal end of the tool.

7. The tool of claim 5, wherein the longitudinally extending arcuate member comprises at least two separate members configured in a clam-shell arrangement operatively connected to one another by a living hinge.

8. The tool of claim 5, wherein a housing is retractably disposed about the cutting mechanism.

9. An apparatus for controlling tissue growth around a catheter comprising:
an annular member configured to at least partially circumscribe a catheter, wherein the annular member includes at least two layers, the at least two layers comprising different materials.

10. The apparatus of claim 9, wherein at least one layer comprises a non-porous material.

11. The apparatus of claim 9, wherein at least one layer comprises a non-absorbable material.

12. The apparatus of claim 9, wherein at least one layer comprises a porous material.

13. The apparatus of claim 9, wherein at least one layer comprises an absorbable material.

14. An apparatus for removing a catheter from a body comprising:
a longitudinally extending arcuate member configured to be disposed about at least a portion of a shaft of a catheter, the member having a distal end and a proximal end;
a first tapered extension disposed on the distal end of the longitudinally extending arcuate member; and
a second tapered extension disposed on a proximal end of the longitudinally extending arcuate member.

15. The apparatus of claim 14, wherein a minimum diameter of the second tapered extension is greater than a maximum diameter of the first tapered extension.

16. The apparatus of claim 14, wherein the longitudinally extending arcuate member comprises at least separate two members configured in a clam-shell arrangement operatively connected to one another by a living hinge.

17. A catheter securement device comprising:
an annular member configured to at least partially circumscribe a catheter, wherein the annular member further comprises a distal band and a proximal band, the two bands being separated a predetermined longitudinal distance along the body of the catheter; and
a plurality of helical members connected to the distal band and the proximal band.

18. The catheter securement device of claim 17, wherein the plurality of helical members comprise a rigid or semi-rigid material.

19. The catheter securement device of claim 17, wherein the plurality of helical members are configured to constrict when axial forces are applied to at least one of the bands.

20. The catheter securement device of claim 17, wherein the distal band is fixed to the body of the catheter.
